(19) United States
(12) Patent Application Publication
(10) Pub. No.: US 2013/0237796 A1
(43) Pub. Date: Sep. 12, 2013

(54) SUPPORTS FOR COMPONENTS IN CATHETERS

(71) Applicants: Anthony Van LE, San Jose, CA (US); Nicholas John RICHARDI, Manteca, CA (US)

(72) Inventors: Anthony Van LE, San Jose, CA (US); Nicholas John RICHARDI, Manteca, CA (US)

(73) Assignee: POINCARE SYSTEMS, INC., Menlo Park, CA (US)

(21) Appl. No.: 13/414,416

(22) Filed: Mar. 7, 2012

(51) Int. Cl. A61B 6/00

(52) U.S. Cl. 600/407

(57) ABSTRACT

A medical device includes an elongate member having a proximal end, a distal end, a body extending between the proximal end and the distal end, and a lumen located within the body. The medical device also includes a tube located inside the lumen, wherein the tube is rotatably supported inside the lumen, a optical waveguide located inside the tube, and a bearing element located in the lumen and disposed between the tube and the body of the elongate member. The elongate member further has a side wall and a region at the side wall for allowing an output light from the optical waveguide to exit therethrough.

### FIELD

This application generally relates to medical imaging, and more specifically, to systems and methods for rotational scanning of internal bodily structures.

### BACKGROUND

Imaging devices may be used to perform imaging at internal region of a human body. Optical coherence tomography (OCT) is an imaging technique that involves rotating a light beam to gather image signals of a target region.

Applicant of the subject application determines that it would be desirable to have a new imaging device with a rotating optical waveguide, and new techniques for rotatably supporting such optical waveguide in an imaging device.

### SUMMARY

In accordance with some embodiments, a medical device includes an elongate member having a proximal end, a distal end, a body extending between the proximal end and the distal end, and a lumen located within the body, a tube located inside the lumen, wherein the tube is rotatably supported inside the lumen, an optical waveguide located inside the tube, and a bearing element located in the lumen and disposed between the tube and the body of the elongate member, wherein the elongate member further has a side wall and a region at the side wall for allowing an output light from the optical waveguide to exit therethrough.

In accordance with other embodiments, a medical device includes a flexible elongate member having a proximal end, a distal end, a body extending between the proximal end and the distal end, and a lumen located within the body, a tube located inside the lumen, wherein the tube is rotatably supported inside the lumen, an optical waveguide located inside the tube, and a plurality of spheres located in the lumen and disposed between the tube and the body of the elongate member, wherein the elongate member further has a side wall and a region at the side wall for allowing an output light from the optical waveguide to exit therethrough.

In accordance with other embodiments, a medical device includes an elongate member having a proximal end, a distal end, a body extending between the proximal end and the distal end, and a lumen located within the body, a tube rotatably supported inside the lumen, wherein the tube has a cross sectional dimension that varies along a longitudinal length of the tube in a periodic manner, and an optical waveguide located inside the tube, wherein the elongate member further has a side wall and a region at the side wall for allowing an output light from the optical waveguide to exit therethrough.

Other and further aspects and features will be evident from reading the following detailed description of the embodiments, which are intended to illustrate, not limit, the invention.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates an imaging probe in accordance with some embodiments;

FIG. 1A illustrates an imaging probe in accordance with other embodiments;

FIG. 1B illustrates an imaging probe in accordance with other embodiments;

FIG. 1C illustrates an imaging probe in accordance with other embodiments;

FIG. 2 illustrates an imaging probe that includes a sheath in accordance with some embodiments;

FIG. 3-7 illustrates different optical systems that may be used in any of the imaging probes of FIGS. 1-2 in accordance with different embodiments;

FIG. 8 illustrates another imaging probe in accordance with other embodiments;

FIG. 9 illustrates an example of a cross section of the imaging probe of FIG. 8 in accordance with some embodiments;

FIG. 10 illustrates another example of a cross section of the imaging probe of FIG. 8 in accordance with other embodiments;

FIG. 11 illustrates another imaging probe in accordance with other embodiments;

FIG. 12 illustrates another imaging probe in accordance with other embodiments;

FIG. 13A illustrates another imaging probe in accordance with other embodiments;

FIG. 13B illustrates another imaging probe in accordance with other embodiments; and

FIG. 14 illustrates another imaging probe in accordance with other embodiments.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

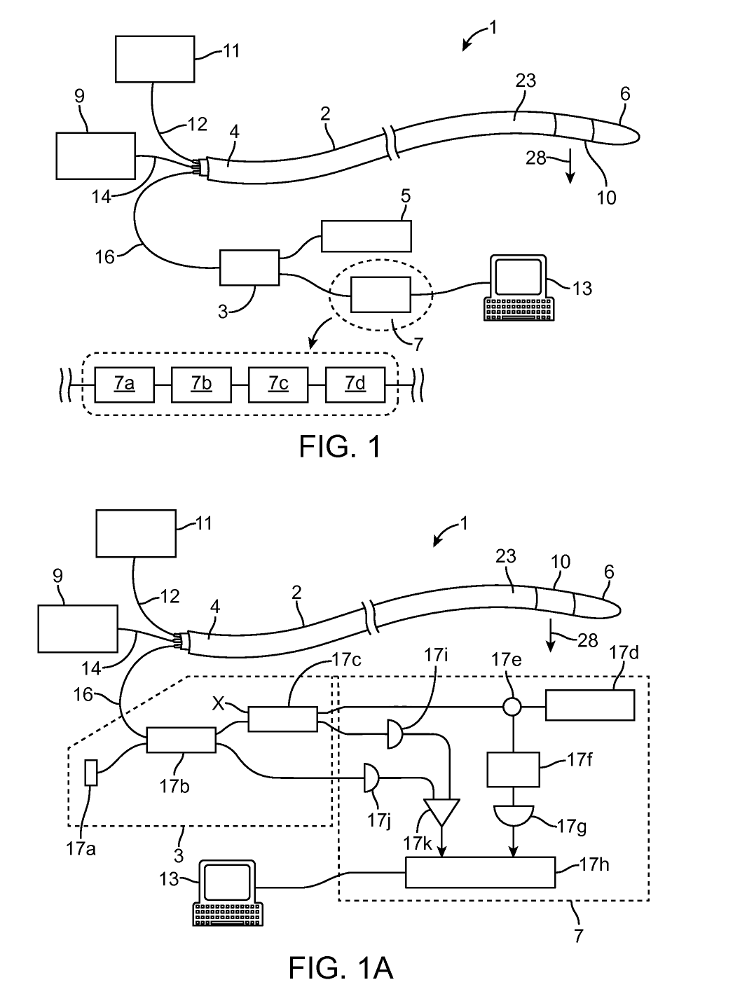

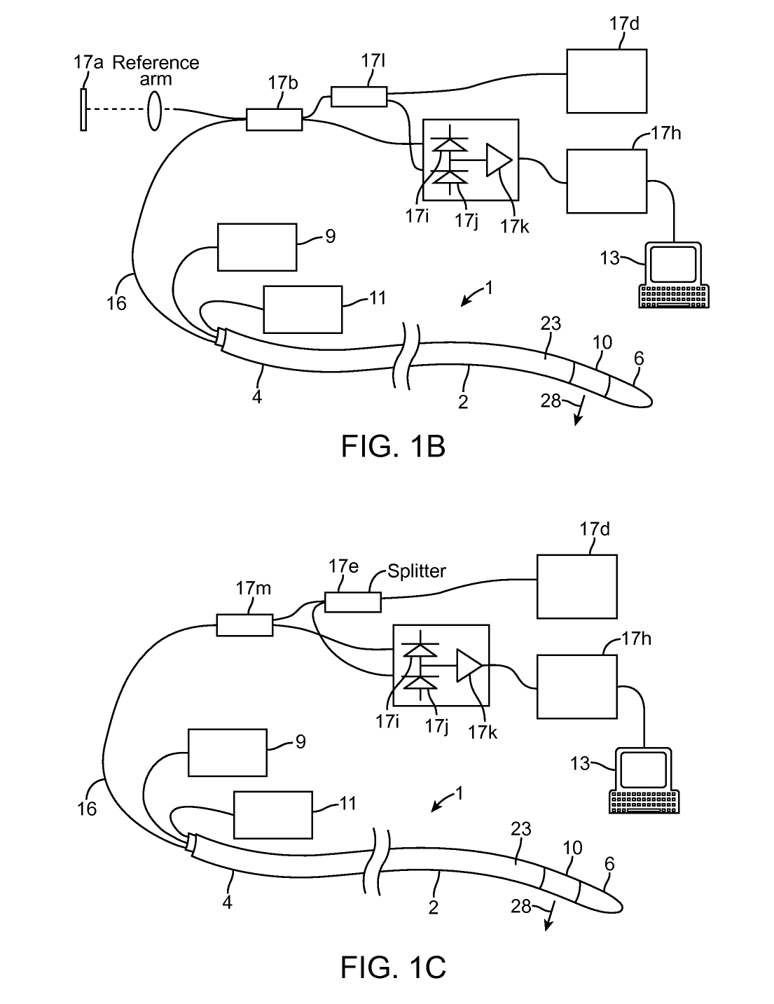

Referring to FIG. 1, an imaging probe 1 is shown in accordance with some embodiments. The imaging probe 1 may have an outer dimension that is anywhere between 50 micron to 50 mm, and more preferably, between 0.5 mm to 10 mm, and even more preferable between 0.4 mm to 1.5 mm (such as 1 mm). Thus, the imaging probe 1 may be placed at different regions inside a body to obtain images. By means of non-limiting examples, the regions may include the aorta, colon, ear canal, esophagus, fallopian tube, blood vessel (vein, artery), passage way in a lung, etc. In other embodiments, the imaging probe 1 may have other outer dimensions that are different from the ranges described above.

In different embodiments, the imaging probe 1 may be configured to perform different types of imaging, such as optical coherence tomography (also known as optical frequency domain imaging), mulitphoton imaging, confocal imaging, Raman spectroscopy, spectroscopy, scanning imaging spectroscopy, and Raman spectroscopic imaging. In other embodiments, the imaging probe 1 may perform other types of imaging.

The imaging probe 1 has an elongated tube 2 with a proximal end 4, a distal end 6, and a body 23 extending between the proximal end 4 and the distal end 6. The imaging probe 1 also has a transparent region 10 located between the proximal end 4 and the distal end 6 such that a focused light beam 28 can pass therethrough from inside the imaging probe 1 in a radial direction to perform an image scanning. The region 10 may have an arc or ring configuration, which allows the beam 28 to exit through the region 10 at different angular positions. The region 10 also allows light (e.g., light provided from the probe 1 and reflected from a tissue) from outside the imaging probe 1 to enter into the imaging probe 1. The region 10 may be completely transparent in some embodiments. In other embodiments, the region 10 may be partially transparent, as long as it can allow some light to pass therethrough in both directions. The imaging probe 1 also includes a fluid connection 12, an electrical connection 14, and an optical connection 16, all located at the proximal end 4.

The fluid connection 12 is configured to couple to a fluid source 11 (such as a saline filled syringe or IV bag) to provide for fluid for flushing the distal end of the imaging probe 1 during use. In such cases, the distal end of the imaging probe 1 may include a flush port in fluid communication with the fluid connection 12. The flush port may aim at the transparent region 10 of the imaging probe 1. In other embodiments, the fluid may be ringers lactate solution, radio-opaque fluid (such as Visopaque™,) or other agent. During imaging, there may be blood flow, and the blood cells may scatter the light, and/or may act as little particles that block the light beam, causing the image quality to drop down significantly. The flush port is advantageous because it allows the distal end of the imaging probe 1 to be cleaned during use. In other embodiments, the fluid connection 12 may be in fluid communication with a lumen in the imaging probe 1. In such cases, the fluid source 11 may provide fluid through the connection 12 to flush fluid to clear the lumen, and/or to partially or completely dilute blood to reduce light scattering caused by blood cells thereby allowing capture of higher quality images. In further embodiments, the fluid connection 12 may be connected to a suction device, which provides a vacuum suction for aspiration to suck materials (e.g., fluid, object, etc.) out of the lumen. The fluid connection 12 is illustrated as being on the probe 1, but in other embodiments, the fluid connection 12 may be on a sheath that surrounds the probe 1.

In the illustrated embodiments, the imaging probe 1 is a part of an imaging system that includes a module 3 comprising of an interferometer, a laser source 5, a processing module 7, and a user interface 13. In other embodiments, any one or a combination of the components 3, 5, 7, and 13 may be considered component(s) of the imaging probe 1. The module 3 is optically coupled to the imaging probe 1 through the optical connection 16 during use. The laser source 5 is configured to provide a broadband input light to the module 3. In the illustrated embodiments, the input light is in an infrared range. In some embodiments, the input light has a center wavelength that is anywhere between 100 nm and 11000 nm, and more preferably, anywhere between 1000 nm and 2000 nm, and even more preferably anywhere between 1100 nm and 1600 nm (such as 1310 nm). In other embodiments, the input light may have other wavelengths. The module 3 passes the input light to an optical waveguide that transmits the input light to the inside of the imaging probe 1. The input light is processed optically (e.g., focused, collimated, reflected, etc.) inside the imaging probe 1, and the processed input light is output through region 10 of the imaging probe 1 as an output light. In the illustrated embodiments, the output light has a wavelength that is anywhere between 100 nm and 11000 nm, and more preferably anywhere between 500 nm and 1500 nm, and even more preferably anywhere between 12100 nm and 1400 nm (such as 1310 nm). In other embodiments, the output light may have other wavelengths. It should be noted that the term “light” or similar terms (such as “light beam”) is not limited to non-visible light, and may refer to any radiation in different wavelengths, which may or may not be visible.

The output light from the imaging probe 1 impinges onto a tissue within a patient, and is reflected from the tissue. The reflected light from the tissue is then captured by the probe 1 through region 10, is optically processed inside the imaging probe 1, and is then transmitted by optical waveguide back to the module 3. The module 3 passes the light signal from the probe 1 to the processing module 7. The processing module 7 detects and processes the signal, and transmits it to the user interface 13. In the illustrated embodiments, the processing module 7 includes one or more photodetector(s) 7a, a signal amplifier or conditioner with an ant-alias filter 7b, an A/D converter 7c, and a Fast Fourier Transform (FFT) processor 7d. The photodetector(s) 7a is configured to detect light containing the depth encoded interferogram from module 3, and convert the light to electrical signal(s). The electrical signals are further conditioned and amplified by the component 7b to be suitable for use by the ND converter 7c. Once the signal is converted from the analog domain to digital domain by the A/D converter 7c, the FFT processor 7d converts the depth encoded electrical interferogram signal via FFT to a depth resolved signal for each point scanned by the imaging probe 1. The FFT processor 7d maybe a discrete processing board, or maybe implemented by a computer. The user interface 13 may be a computer (as illustrated), a hand-held device, or any of other devices that is capable of presenting information to the user. The user interface 13 reconstructs the image from the FFT processor 7d and display a result (e.g., an image) of the processing in a screen for the user's viewing.

The delivering of output light by the imaging probe 1, and the receiving of reflected light by the imaging probe 1, may be repeated at different angles circumferentially around the probe 1, thereby resulting in a circumferential scan of tissue that is located around the imaging probe 1. In some embodiments, one or more components within the distal end of the probe 1 are configured to rotate at several thousand times per minute, and the associated electronics for processing the light signals are very fast, e.g., has a sample rate of 180,000,000 times a second. In other embodiments, the one or more components within the distal end of the probe 1 may rotate at other speeds that are different from that described previously. Also, in other embodiments, the associated electronics for processing the light signals may have a data processing speed that is different from that described previously.

The electrical connection 14 may be used to control functions of the imaging probe 1, as well a providing power to magnetic coils to turn a rotor that is in, or coupled to, the probe 1. In some embodiments, the electrical connection 14 may be connected to one or more sensors to sense position, velocity, acceleration, jerk, etc., of a rotor that is in, or coupled to, the probe 1.

The imaging system also includes a control 9 electrically coupled to the imaging probe 1 through the electrical connection 14. In some embodiments, the control 9 may be used to control a positioning of one or more optical components located inside the imaging probe 1. For example, in some embodiments, the control 9 may have a manual control for allowing a user to control a turning (e.g., amount of turn, speed of turn, angular position, etc.) of a beam director (e.g., a mirror or a prism) which directs the light beam 28 to exit through the region 10 at different angles.

In other embodiments, the control 9 may having a manual control for allowing a user to move one or more lens inside the imaging probe 1 so that a focusing function may be performed. In further embodiments, the control 9 may have a switch which allows a user to select between manual focusing, or auto-focusing. When auto-focusing is selected, the imaging system will perform focusing automatically.

In still further embodiments, the control 9 may also includes one or more controls for allowing a user to operate the imaging probe 1 during use (e.g., to start image scanning, stop image scanning, etc.).

In further embodiments, the imaging probe 1 is flexible and is steerable using the control 9. In such cases, the imaging probe 1 may include a steering mechanism for steering the distal end 6 of the imaging probe 1. For example, the steering mechanism may include one or more wires coupled to the distal end 6 of the imaging probe 1, wherein tension may be applied to any one of the wires using the control 9. In particular, the control 9 may include a manual control that mechanically couples to the wire(s). During use, the user may operate the manual control to apply tension to a selected one of the wires, thereby resulting in the distal end 6 bending in a certain direction.

The imaging probe 1 may be implemented using different devices and/or techniques. FIG. 1A illustrated an example of how the components 3, 7 of the imaging probe 1 may be implemented in accordance with some embodiments. In the illustrated embodiments, the module 3 includes optical waveguide couplers 17b and 17c forming an interferometer. Reference mirror 17a is connected to reference arm of the interferometer, while the sample arm of the interferometer is connected to the imaging probe 1 through connection 16. Light from laser 17d is transmitted to a splitter 17e, which divides a portion of the light from the laser 17d for transmission to the module 3, while the other portion of the light is diverted to a reference clock interferometer 17f. At the module 3, the light from the laser 17d is received at the coupler 17c, and is then transmitted to the coupler 17b, wherein part of the light is passed to the reference mirror 17a, and the rest is passed to the imaging probe 1. The light at the reference mirror 17a is reflected back to the coupler 17b, which divides the light so that a portion of it goes to the coupler 17c and to the photo detector 17i, and another portion of it goes to the photo detector 17j. The light delivered to the probe 1 exits from the region 10 of the imaging probe 1 and strikes a sample. The imaging probe 1 then detects the reflected light back from the sample, and optically communicates the reflected light through imaging probe 1 and module 3, where the path length difference creates an interferogram containing the depth encoded information which is detected by photo detectors 17i and 17j. In particular, the light from the sample is transmitted to the coupler 17b, which divides the light so that a portion of it goes to the coupler 17c and to the photo detector 17i, and another portion of it goes to the photo detector 17j. Photodetectors 17i and 17j are optically communicated to module 3 and are configured for providing balanced signal detection using differential amplifier 17k. Thus, for every light signal provided by the source 17d, the differential amplifier 17k receives a reflected from the reference mirror 17a, and another signal from the light sampled at the distal end of the probe 1. The signal from the differential amplifier 17k is then digitized by the A/D converter 17h. Reference clock interferometer 17b is optically communicated to photo detector 17g to covert the optical clocking signals to electrical signals. In the illustrated embodiments, the interferometer 17f may be implemented using a Fabry Perot interferometer or Mach-Zehnder interferometer. In other embodiments, the interferometer 17f may be implemented using other devices. The electrical clocking signals from 17g are used to provide the clocking signal in even wavenumber space for the A/D converter 17h, which digitizes the analog signals and converts them into the digital domain for further processing. In the illustrated embodiments, the user interface 13 includes a computer, which may be used to perform FFT on the signals from the A/D converter 17h. The computer then reconstructs one or more images for display at a screen of the user interface 13. In some embodiments, the user interface 13 reconstructs the images by placing the processed signals from FFT into a rectangular array, which is then mapped to polar coordinates representing the radial scan performed by the imaging probe 1. The data is then compressed logarithmically to compress the dynamic range of the signal such that it is easily perceived by the user, which is then displayed as an intensity mapped image showing the fully reconstructed image for the user to view. The computer may also be used to perform further signal processing and/or image processing, if desired. Alternatively FFT, signal processing, and/or image reconstruction may be performed using a separate module(s) or device(s). The image(s) at the user interface 13 may then be used for diagnostic and/or treatment purposes. It should be noted that the imaging probe 1 is not limited to the example illustrated, and that in other embodiments, the imaging probe 1 may have different configurations.

It should be noted that the imaging system is not limited to the example described previously, and that in other embodiments, the imaging system may have other configurations. FIG. 1B illustrates another imaging system, which is similar to that shown in FIG. 1A, except that the coupler 17b and circulator 171 are used to form a Michelson interferometer, similarly having reference and sample arms whereby reference arm is optically communicated to the mirror 17a, and sample arm is optically communicated to the imaging probe 1. FIG. 1C illustrates another imaging system, which is similar to that shown in FIG. 1A, except that it includes a circulator 17m optically communicated to the imaging probe 1 to form a common path interferometer, whereby both reference and sample arm optical beam paths are combined, and where the reference mirror 17a is now present within the optical beam path within the imaging probe 1.

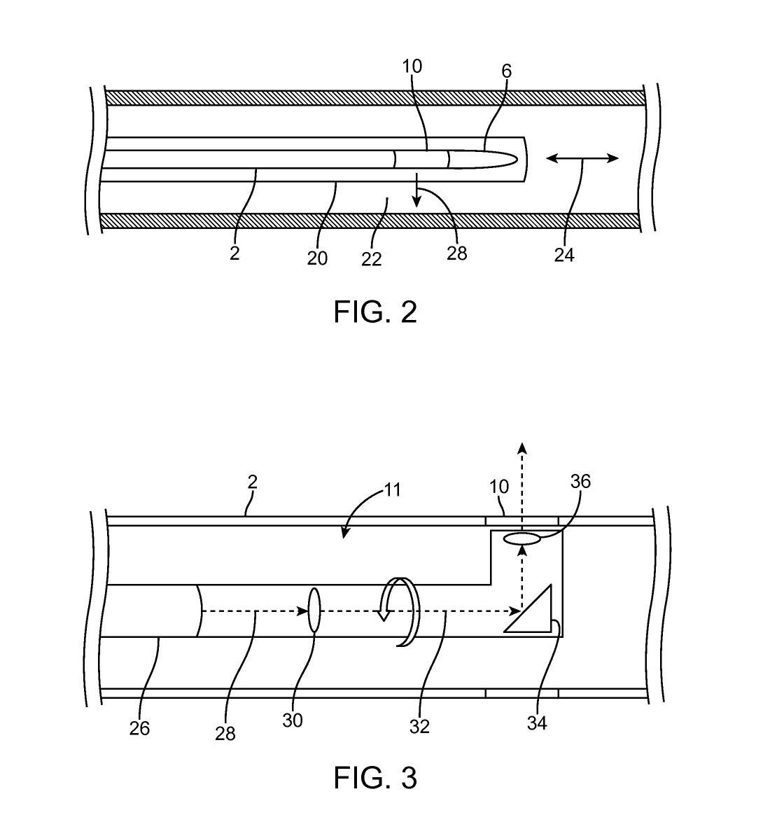

As shown in FIG. 2, in some embodiments, the imaging probe 1 of FIG. 1 may be placed within an elongated sheath 20. In some embodiments, part of the sheath 20 along its length may have a transparent region (similar to region 10 on the probe 1) so that light from the imaging probe 1 may exit through the transparent region of the sheath 20. In such cases, the length of the transparent region at the sheath 20 may be longer than the transparent region 10 at the imaging probe 1, so that when the probe 1 is placed at different positions relative to the sheath 20, light from the probe 1 can exit through the transparent region at the sheath 20. In other embodiments, the entire sheath 20 may be transparent. During use, the imaging probe 1 within the elongated sheath 20 can be placed in a narrow void or lumen 22 inside a patient to perform imaging using the focused light beam 28. The imaging probe 1 can be moved along the inside of the elongated sheath 20 (shown by arrow 24) to allow for imaging of the narrow void or lumen 22 along a preferred region. The sheath 20 is advantageous in that it prevents the probe 1 from rubbing against tissue during use. In other embodiments, the sheath 20 may not have any transparent region. In such cases, after the sheath 20 is desirably placed within the lumen 22 inside the body, the probe 1 can be deployed out of an opening at a distal end of the sheath 20.

As discussed, the imaging probe 1 allows the light beam 28 to exit through the region 10 at different angles. Such may be accomplished by turning an optical waveguide 26 (e.g., a fiber optic) and a beam director located inside the imaging probe 1. FIG. 3 illustrates an optical system 11 located within the imaging probe 1 in accordance with some embodiments, the optical system 11 includes an optical waveguide 26, a collimation lens 30, a beam director 34, and a focusing lens 36. Components of the optical system 11 may be placed at a distal region of the probe 1. The optical waveguide 26 is configured to provide a light beam 28, which is then optically processed by the collimation lens 30, the beam director 34, and the focusing lens 36. The processed light beam 28 then exits through the transparent region 10 of the imaging probe 1. The optical waveguide 26 may be an optical fiber (e.g., a single mode optical fiber, a multimode optical fiber, a fiber bundle, etc.), a hollow reflective capillary tube, a capillary tube with an inside diameter coated with at least one dielectric coating, a photonic crystalline fiber (also known as a Holley fiber), or any optical transmitter that is capable of transmitting or directing light. The optical waveguide 26 aligns with the collimation lens 30, which collimates the diverging light from the waveguide 26. In some embodiments, the collimation lens 30 may be a plano convex lens that changes a diverging light to a collimated light having a parallel configuration. In other embodiments, the collimation lens 30 may be a bi-convex lens that not only changes a diverging light to have a parallel configuration, but also focuses the light.

Also, in the illustrated embodiments, the collimation lens 30 is aligned with the beam director 34. A focusing lens may be placed between the collimation lens 30 and the beam director 34. Alternatively, the focusing lens may be placed after the beam director 34. The beam director 34 may be an optical component that is capable of changing a path of a light. For example, the beam director 34 may be a mirror, or a prism. The beam director 34 is configured to direct (e.g., deflects) the light so that the light changes direction. In the illustrated embodiments, the light leaving the beam director 34 travels in a direction that is 90° from the original path of the light. In other embodiments, the light leaving the beam director 34 may travel in a direction that forms other angles relative to the original path. As shown in the figure, the beam director 34 is next to the transparent region 10 at a position along a longitudinal axis of the imaging probe 1. This allows light leaving the beam director 34 to exit through the transparent region 10. The light beam 28 is directed by the beam director 34 radially from the longitudinal axis of optical waveguide 26, and is optically communicated to the focusing lens 36, which focuses the light beam 28 to form an output light. As shown in the figure, the optical waveguide 26, the beam director 34 and the focusing lens 36 are configured to rotate about the axis 32 of the waveguide 26, so that the light beam 28 may exit through the region 10 at different angular positions.

The output light provided by the probe 1 impinges on tissue, and is reflected back towards the imaging probe 1. The reflected light enters through the transparent region 10, and is collimated by the focusing lens 36. The light is then directed by the beam director 34 towards the lens 30. Lens 30 then focuses the light, which is then transmitted to the optical waveguide 26. The optical waveguide 26 transmits the light to components 3 and 7 for processing the light signal. Thus, as illustrated in the above embodiments, the collimation lens 30 has bi-directional properties (i.e., collimation in one direction, and light-focusing in the other direction), and the focusing lens 36 also has bi-directional properties (i.e., light-focusing in one direction, and collimation in the other direction).

Accordingly, as used in this specification, the term “collimation lens” is not limited to an optical device that only performs collimation, and may refer to any optical device that is capable of performing other functions, such as, light focusing. Similarly, as used in this specification, the term “focusing lens” is not limited to an optical device that only performs light focusing, and may refer to any optical device that is capable of performing other functions, such as, light collimation. Also, in any of the embodiments described herein, any of the optical components may have uni-directional property or bi-directional properties.

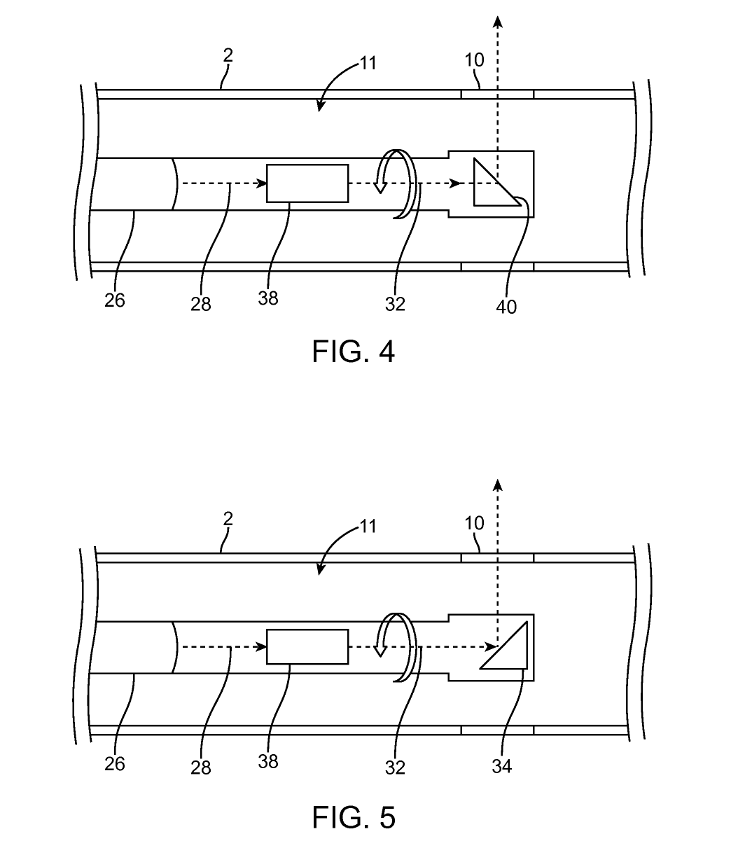

The optical system 11 is not limited to the example described previously, and may have other configurations in other embodiments. As shown in FIG. 4, in other embodiments, the optical system 11 may include an optical waveguide 26 that transmit the light beam 28 to an gradient index lens 38. The gradient index lens 38 may be configured to convert the diverging light beam 28 to a parallel light beam 28, and/or to focus the light beam 28. The gradient index lens 38 is advantageous in that it is configurable to provide a desired optical output. As shown in figure, the light beam 28 is received by a beam directing prism 40 (another example of the beam director 34), which directs at least some of the light beam 28 to exit from the region 10 at the imaging probe 1. The optical waveguide 26 and the beam directing prism 40 are configured to rotate around the axis 32. Beam directing prism 40 directs light beam 28 radially outward from the axis of optical waveguide 26. In particular, the light beam 28 is directed by the beam director 34 radially from the longitudinal axis of optical waveguide 26. In some embodiments, the imaging probe 1 may further include a focusing lens (like the focusing lens 36 shown in FIG. 3). In such cases, the prism 40 is optically communicated to the focusing lens 36, which focuses the light beam 28 provided from the prism 40 to form an output light. In other embodiments, the imaging probe 1 of FIG. 4 may optionally further include the focusing lens 36 as similarly discussed with reference to FIG. 3.

In other embodiments, instead of the prism 40, the beam director 34 of may be a mirror (FIG. 5). The embodiments of FIG. 5 may optionally include a focusing lens (like the focusing lens 36 of FIG. 3).

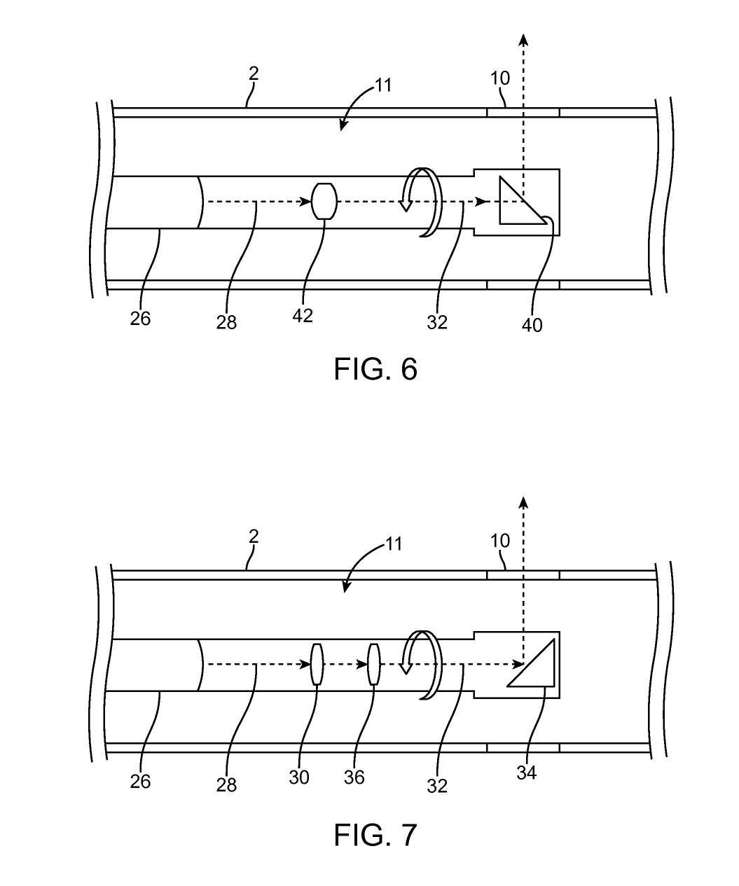

Also, in other embodiments, instead of the gradient index lens 38, the imaging probe 1 may include a finite conjugate lens 42 (FIG. 6). The finite conjugate lens 42 provides collimation and focusing of light using one lens. Alternatively, instead of the finite conjugate lens 42, two separate lenses may be used, wherein one is for collimation of light, and the other one is for focusing the light. The operation of the embodiments of FIG. 6 is similar to that described with reference to FIG. 4. In other embodiments, instead of the prism 40 shown, the imaging probe 1 of FIG. 6 may have a mirror as the beam director 34. Also, in other embodiments, the imaging probe 1 may optionally include a focusing lens (like the focusing lens 36 of FIG. 3).

In further embodiments, instead of having the focusing lens 36 at the downstream side of the beam director 34, the focusing lens 36 may be placed upstream to the beam director 34 (FIG. 7). In such cases, the collimation lens 30 is configured to change a diverging light 28 to have a parallel configuration. The parallel light beam 28 reaches the focusing lens 36 and is focused by the focusing lens 36. The focused light beam 28 reaches the beam director 34 (illustrated as a mirror in the example), and is directed to exit through region 10 of the imaging probe 1. In other embodiments, the beam director 34 may be a prism. Also, in other embodiments, an additional focusing lens may be placed downstream from the beam director 34 to further focus the light beam 28 (such as that described with reference to FIG. 3).

Also, in any of the embodiments of the imaging probe 1 described herein, the collimation lens may be implemented using micro optic(s), fiber lens, other any of other known devices, to collimate the beam. As discussed herein, the collimation optics may be located in the axis that is coincident with the axis of the transmitted light provided by the optical waveguide 26. Also, in any of the embodiments of the imaging probe 1 described herein, the focusing optics may be located in line with the collimation optics, or may be located 90 degrees (or at other angles relative) to the emitted light axis from the optical waveguide 26. Furthermore, in any of the embodiments of the imaging probe 1 described herein, the beam director 34 may include a concave mirror, which not only direct the light beam at a certain angle (e.g., 90°), but also to focus it as well. In still further embodiments, any of the embodiments of the imaging probe 1 may include optical device(s) that function as filter(s), such as notch, shortpass, longpass, bandpass, fiber Bragg gratings, optical gratings. Such optical device(s) may be placed in line with the optics described herein to further provide optical manipulation of the light as it is emitted or detected by the probe 1 for optical enhancement. In any of the embodiments of the imaging probe 1 described herein, the optical components in the probe 1 may be configured (e.g., positioned, placed, arranged, etc.) to allow bidirectional coupling of light to and from the proximal and distal ends of the probe 1.

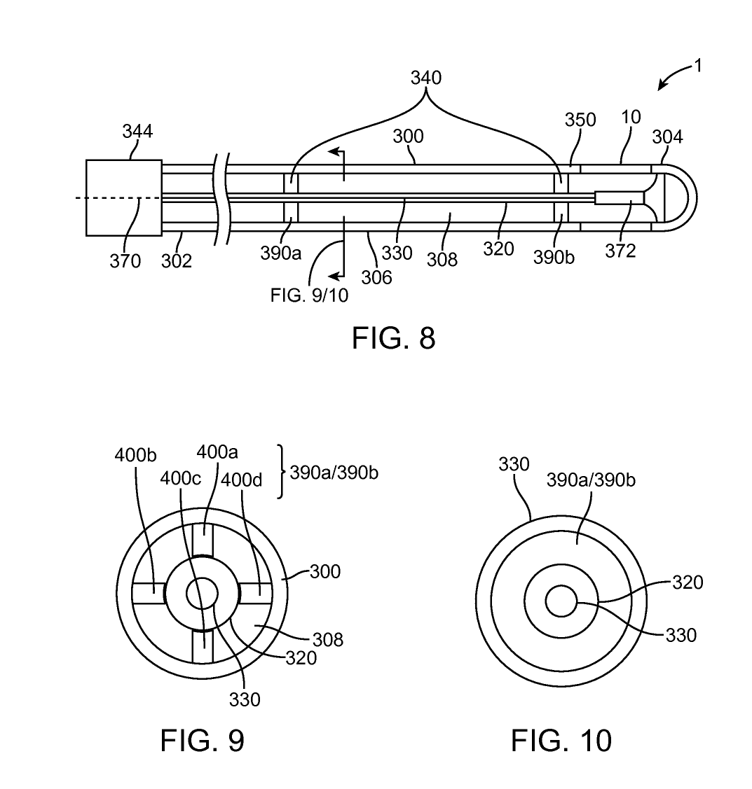

As discussed, the optical waveguide in the imaging probe 1 is configured to rotate during use. Various techniques may be employed to rotatably support the optical waveguide inside the imaging probe 1. FIG. 8 illustrates an imaging probe 1 in accordance with some embodiments. The imaging probe 1 may be any of the imaging probes 1 described with reference to FIGS. 1-1B. In some embodiments, the imaging probe 1 may be a flexible catheter. In other embodiments, the imaging probe 1 may be rigid. The imaging probe 1 includes an elongate member 300 having a proximal end 302, a distal end 304, a body 306 extending between the proximal end 302 and the distal end 304, and a lumen 308 located within the body 306. The elongate member 300 may be the elongate tube 2 described with reference to FIGS. 1-1B. The elongate member 300 may be made from polyurethane, nylon, polyethylene, FEP, PET, Pebax, polymers, or any of other materials. The imaging probe 1 also includes a tube 320 located inside the lumen 308, wherein the tube 320 is rotatably supported inside the lumen 308, an optical waveguide 330 located inside the tube 320, and a bearing element 340 located in the lumen 308 and disposed between the tube 320 and the body 306 of the elongate member 300. The elongate member 300 further has a side wall 350 and a region 10 at the side wall 350 for allowing an output light from the optical waveguide 330 to exit therethrough.

In some embodiments, the tube 320 may be a hypotube that is made from a metal or alloy such as stainless steel, nitinol, kovar, invar, or cobalt-chromium alloy, etc. In other embodiments, the tube 320 may be made from other materials. Also, in some embodiments, the tube 320 may have a cross sectional dimension that is anywhere from 100 microns to 500 microns (e.g., 300 microns). In other embodiments, the tube 320 may have a cross sectional dimension that is less than 100 microns, or larger than 500 microns.

As shown in the figure, the imaging probe 1 also includes a motor 344 coupled to the proximal end 302 of the elongate member 300 for turning the tube 320 and the optical waveguide 330 relative to the body 306 of the elongate member 300. In particular, during use, the motor 344 rotates the tube 320 and the optical waveguide 330 relative to the body 306 of the elongate member 300 about the longitudinal axis 370. The optical waveguide 330 delivers the output light to an optical system 372, which directs the output light from the optical waveguide 330 to exit laterally through the region 10. The optical system 372 is rotatably supported at its distal end by a support 374, which is fixedly secured to the elongate member 300. The optical system 372 may be any of the optical systems 11, or may include any subset of components in any of the optical systems 11, described with reference to FIGS. 3-7. The optical system 372 may include a mirror, a prism, a beam splitter, or any of other optical elements that is capable of changing a direction of a light path. The optical system 372 is coupled to the tube 320 so that as the tube 320 and the optical waveguide 330 rotate, the optical system 372 also rotates together with the tube 320 and the optical waveguide 330. As used in this specification, the term “optical system” may refer to one or more optical components.

The bearing element 340 includes a first bearing 390a, and a second bearing 390b that are disposed in the lumen 308 between the tube 320 and the body 306 of the elongate member 300. The bearings 390a, 390b are located at different locations along a longitudinal length of the elongate member 300. Although only two bearings 390a, 390b are shown in the example, in other embodiments, there may be more than two bearings 390 disposed at different locations along the longitudinal length of the elongate member. Also, in other embodiments, there may be only one bearing 390. Thus, as used in this specification, the term “bearing element” may refer to one or more bearing components.

In some embodiments, the bearings 390a, 390b may be fixedly secured to an inner wall of the body 306 of the elongate member 300. In such cases, the tube 320 is rotatable relative to the bearings 390a, 390b. In other embodiments, the bearings 390a, 390b may be fixedly secured to an outer wall of the tube 320. In such cases, the elongate member 300 is rotatable relative to the bearings 390a, 390b.

FIG. 9 illustrates an example of a cross section of the device 1 of FIG. 8 in accordance with some embodiments. As shown in the figure, the bearing 390a/390b may include a plurality of supports 400a-400d that are circumferentially disposed around the tube 320. Although four supports 400a-400d are shown, in other embodiments, there may be more than four supports or less than four supports (e.g., two or three supports).

FIG. 10 illustrates another example of a cross section of the device 1 of FIG. 8 in accordance with other embodiments. As shown in the figure, the bearing 390a/390b may be in a form of a ring that surrounds the tube 320. The embodiments of FIG. 9 may be more advantageous than the embodiments of FIG. 10 because the plurality of supports 400 reduces the amount of contact area between the bearing 390a/390b and the tube 320. However, either embodiments of FIG. 9 or FIG. 10 may be used.

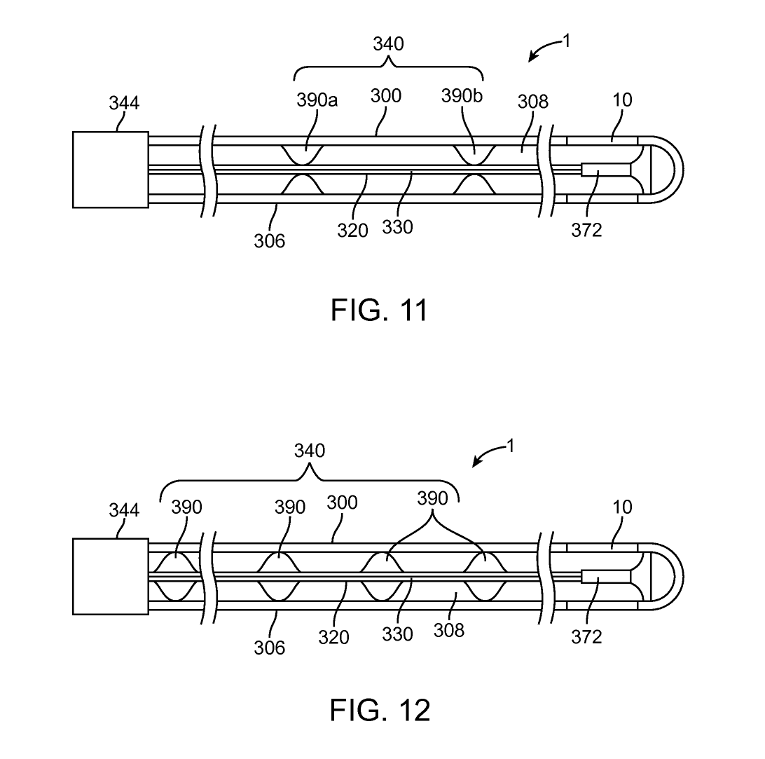

As shown in FIG. 8, the bearing 390a/390b has a constant thickness. However, in other embodiments, the bearing 390a/390b may have a variable thickness. For example, as shown in FIG. 11, the bearing 390a/390b in the device 1 may have a variable thickness that varies as a function of radial distance measured from the inner surface of the body 306 of the elongate member 300. In the illustrated embodiments, the bearings 390a, 390b are secured to an inner wall of the body 306 of the elongate member 300. Each of the bearings 390a, 390b has a thickness that decreases as a function of radial distance measured from the inner surface of the body 306 towards the central part of the lumen 308. Such configuration may be desirable because it reduces the amount of contact area between the bearings 390a, 390b and the tube 320, thereby reducing an amount of friction therebetween as the tube 320 is rotated relative to the body 306. In the embodiments of FIG. 11, each of the bearings 390a, 390b may have a plurality of supports (like that shown in FIG. 9—in which case, each of the supports has a thickness that varies as a function of distance measured radially from the tube), or may have a ring configuration (like that shown in FIG. 10). In some cases, the bearings 390a, 390b with variable thickness may be implemented using jewel bearings.

In other embodiments, the bearings 390 may be fixedly secured to the outer surface of the tube 320 (FIG. 12). During use, the bearings 390 will rotate together with the tube 320 relative to the body 306 of the elongate member 300. As shown in the figure, each of the bearings 390 in the device 1 may have a variable thickness that varies as a function of radial distance measured from the inner surface of the body 306 of the elongate member 300. In the illustrated embodiments, each of the bearings 390 has a thickness that decreases as a function of radial distance measured from the outer surface of the tube 320 towards the wall of the body 306. Such configuration may be desirable because it reduces the amount of contact area between the bearings 390 and the body 306 of the elongate member 300, thereby reducing an amount of friction therebetween as the tube 320 is rotated relative to the body 306. In the embodiments of FIG. 12, each of the bearings 390 may have a plurality of supports (like that shown in FIG. 9—in which case, each of the supports has a thickness that varies as a function of distance measured radially from the tube), or may have a ring configuration (like that shown in FIG. 10).

It should be noted that the bearing element 340 is not limited to the example of bearings described, and that the bearing element 340 may be implemented using any types of bearing, such as cartridge bearings, roller bearings, needle bearings, etc. Also, the bearing element 340 may be implemented using any component(s), as long as the component(s) can function to reduce an amount of friction between the tube 320 and the body 306 of the elongate member 300/2.

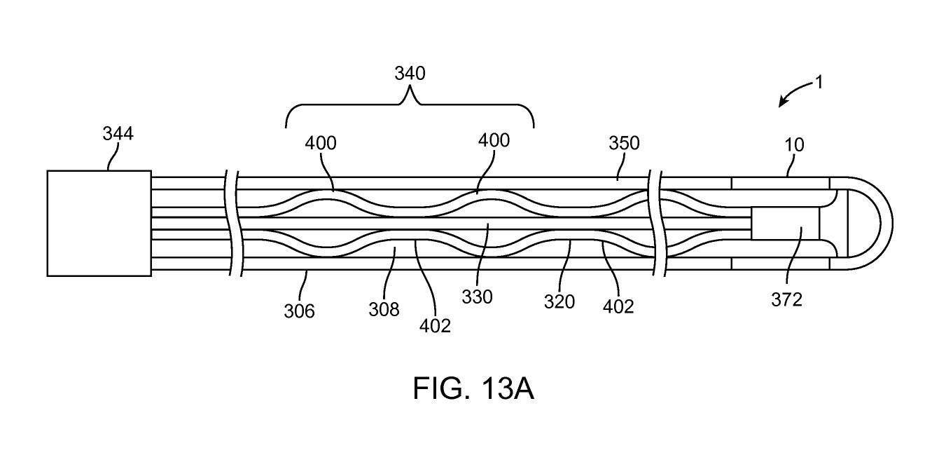

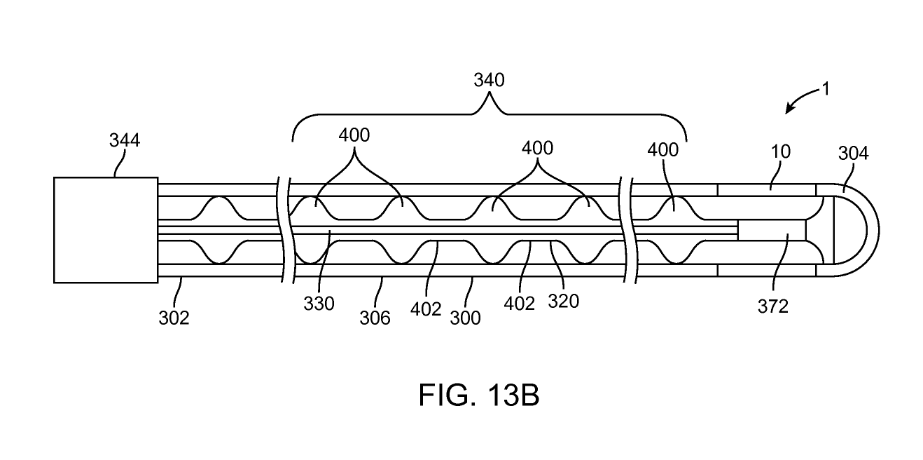

In other embodiments, parts of the tube 320 may be implemented as bearings. FIG. 13A illustrates a variation of the imaging probe 1, particularly showing the tube 320 having a cross sectional dimension that varies along a longitudinal length of the tube 320 in a periodic manner. The imaging probe 1 may be any of the imaging probes 1 described with reference to FIGS. 1-1B. As shown in the figure, the tube 320 includes larger portions 400 and smaller portions 402. The portions 400 of the tube 320 with larger cross sections are engaged with the wall 350 of the elongate member 300, while the portions 402 of the tube 320 with smaller cross sections are engaged with the optical waveguide 330. In the illustrated embodiments, the larger portions 400 of the tube 320 function as bearings to reduce an amount of friction between the tube 320 and the body 306 of the elongate member 300, as the tube 320 is rotated relative to the body 306. Thus, parts of the tube 320 may be considered to be the bearing element 340. In some embodiments, the shape of the tube 320 may be formed using molding techniques. For example, a mold having the desired shape of the tube 320 may be used to form the tube 320. In other embodiments, the shape of the tube 320 may be created by forming a rectilinear tube first, and then expanding (e.g., by heat-expansion technique, by inserting an expandable device into the tube 320 to urge the tube 320 to expand radially outward, etc.) certain parts of the tube 320 to form the large portions 400. In further embodiments, the shape of the tube 320 may be created by forming a rectilinear tube first, and then shrinking (e.g., by mechanically squeezing) parts of the tube 320 to create the small portions 402. In other embodiments, additional bearings may be disposed between the tube 320 and the body 306 of the elongate member 300 to further reduce friction therebetween. For example, in some embodiments, a friction-reducing coating may be disposed on the outer surface of the tube 320, or on an inner surface of the body 306 of the elongate member 300.

FIG. 13B illustrates a variation of the imaging probe 1 of FIG. 13A. The imaging probe 1 of FIG. 13B is similar to that in FIG. 13A, except that the optical waveguide 330 is supported through the length of the tube 320. The shape of the tube 320 may be created by forming a rectilinear tube first, and then shrinking (e.g., by mechanically squeezing) parts of the tube 320 to create the small portions 402.

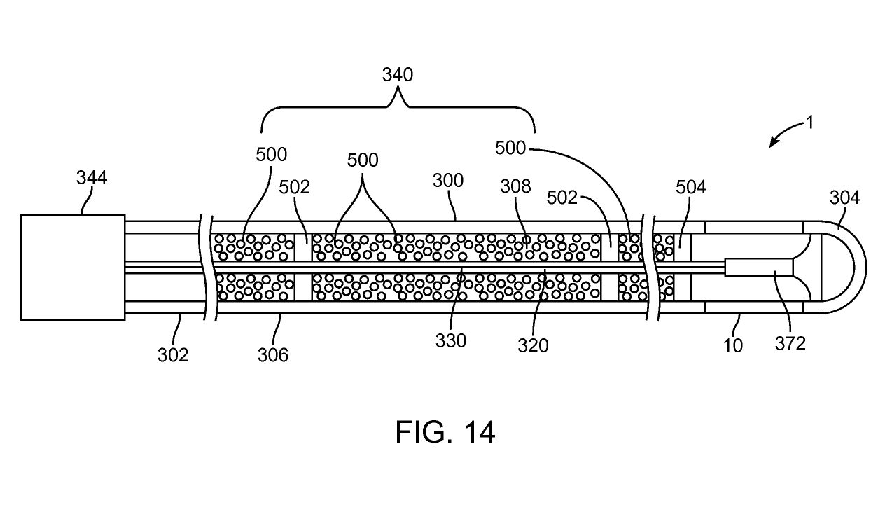

FIG. 14 illustrates another variation of the imaging probe 1, particularly showing another technique for reducing friction between the rotating tube 320 and the body 306 of the elongate member 300. The imaging probe 1 may be any of the imaging probes 1 described with reference to FIGS. 1-1B. As shown in the figure, the imaging probe 1 includes a plurality of spheres 500 that are disposed in the lumen 308 between the tube 320 and the body 306. The spheres 500 are used to implement the bearing element 340 in the illustrated embodiments. The spheres 500 may be microspheres, or nanospheres in some embodiments. In other embodiments, the spheres 500 may be other types of spheres, and may have any dimensions, such as 1-500 nanometers, 0.5 microns to 2000 microns, or even 2000-10000 microns. The spheres 500 may be made from glass, ceramic, polymers, metal, alloys, or any of other materials. The microspheres or nanospheres can have a spherical, a semi-spherical, or even non spherical shape as to allow rolling action to reduce friction. Thus, as used in this specification, the term “sphere” or similar terms (such as “microsphere”, “nanosphere”, etc.) may refer to an object having any shape, and is not necessarily limited to an object having a spherical shape.

In the illustrated embodiments, the imaging probe 1 also includes a plurality of partitions 502 secured to the inner wall of the body 306, and disposed at different locations along a length of the body 306. The partitions 502 separate the spheres 500 into two or more groups of spheres 500. In other embodiments, the partitions 502 may be secured to the outer wall of the tube 320. The partitions 502 are beneficial because they allow the spheres 500 to have a more uniform distribution along the length of the imaging probe 1. For example, in the case in which the spheres 500 are loosely packed in the lumen 308, if there is no partition 502, the spheres 500 may be free to roll towards one end of the imaging probe 1, thereby resulting in the other end of the imaging probe 1 having no spheres 500. The partitions 502 may limit the amount of movement by the spheres 500 in the lumen 308, thereby creating a more uniform distribution of the spheres 500 (compared to the case in which no partition 502 is used).

The partitions 502 are also advantageous because they may provide different characteristics for different portions along the length of the imaging probe 1. For example, in some embodiments, one group of the spheres 500 may be packed more densely than another group of spheres 500 that are separated by the partition 502. This has the effect of stiffening a part (which has more densely packed spheres 500) of the imaging probe 1 relative to another part (which has more loosely packed spheres 500). In some cases, if the distal portion of the imaging probe 1 is desired to be more flexible than a proximal portion (e.g., because the imaging probe 1 may be desired to be steered in more curvy paths within the patient), then the spheres 500 at the distal portion may be packed less dense compared to the spheres 500 at the proximal portion. Also, in other embodiments, one group of spheres 500 may have different size compared to another group of spheres 500 that are separated by the partition 502. In further embodiments, the partitions 502 may not be needed, and the imaging probe 1 does not include any partitions 502.

The spheres 500 function to reduce an amount of friction between the tube 320 and the body 306 of the elongate member 300, while allowing the elongate member 300 to remain flexible. In some embodiments, the fill percentage of the spheres 500 may be anywhere from 0.01% to 97% of the internal volume between tube 320 and body 306 of elongate member 300. As the percentage of fill between internal volume of tube 320 and body 306 increases, the spheres become more densely packed. Also, in some embodiments, the spheres 500 may be loosely packed as long as they can reduce some friction between the tube 320 and the body 306 of the elongate member 300.

In some embodiments, the spheres 500 may be mixed together with a fluid, such as a liquid lubricant, for allowing the spheres 500 to slide easily relative to each other. Alternative, the fluid is not required.

Also, in some embodiments, the spheres 500 may all have the same size. In other embodiments, the spheres 500 may have different sizes. For example, in other embodiments, there may be a distribution of sizes for the spheres 500 ranging from 10 nm to 1 mm. The distribution of sizes may have other ranges that are different from the above example in other embodiments.

As shown in the illustrated embodiments, the imaging probe 1 further includes a blocker 504 proximal to the region 10 for preventing the spheres 500 from travelling distally to interfere with the delivery of light through the region 10. The blocker 504 is illustrated to be in a form of a wall in the illustrated embodiments. In other embodiments, the blocker 504 may have other configurations (e.g., shapes and/or sizes). For example, in other embodiments, the blocker 504 may have a block-like configuration.

It should be noted that the imaging probe 1 is not limited to the examples of the configuration of lenses described previously, and that the imaging probe 1 may have other types of lenses and/or other combination of optical components in other embodiments. For example, in other embodiments, in addition to, or instead of, any of the above optical components, the imaging probe 1 may include axicons, phase mask lenses, Fresnel lenses, aspheric lenses, or combination thereof, to process light in a desired manner (such as focusing, defocusing, collimation, filtering, etc.). Thus, in any of the embodiments of the imaging probe 1 described herein, the optical components may have different configurations (e.g., shape, size, location, arrangement, etc.). Also, in any of the embodiments of the imaging probe 1 described herein, any of the optical components may rely on different way(s) to process light other than refraction.

As discussed, in some embodiments, the elongate member 300 of the imaging probe 1 may be flexible. In other embodiments, the elongate member 300 of any of the embodiments of the imaging probe 1 described herein may have a variable stiffness along the length of the imaging probe 1. For example, in some embodiments, the stiffness of the imaging probe 1 where the motor 344 is located may be higher than the stiffness of other section of the imaging probe 1. In other embodiments, the stiffness of the probe 1 between the proximal and distal ends 4, 6 may be varied to allow or restrict flexibility of the probe 1 to advantageously gain additional nimbleness or control in positioning the probe 1 in narrow and curved voids such as vascular lumens or the GI tract of the human body. In further embodiments, the elongated member 300 of the imaging probe 1 may be rigid.

The stiffness variation of a probe 1 may be implemented in a variety of methods, such as by varying the elasticity of the probe material, and/or by placing braiding or fiber reinforcement within the wall of the probe 1 at certain desired location(s). In any of the embodiments described herein, the probe 1 may be made from a flexible material or polymer material, but may also be made from metal or glass if desired and reinforced with metal or polymer fibers.

Also, in any of the embodiments of the imaging probe 1 described herein, silver active micro particles or nanoparticles may be coated on the surface of the imaging probe 1, or embedded into the wall of the probe 1, such that silver ions are released free from the catheter probe 1, or are present at the probe's surface to provide for anti-bacterial properties. In other embodiments, the probe 1 may be coated with an antibiotic coating to prevent bacterial infection. This antibiotic coating may have a single antibiotic agent, or a combination of antibiotics to prevent an array of different types of bacterial infections.

The optical waveguide 330 that transmits light within the probe 1 may be a single mode or multimode fiber. It is possible that there can be many of these optical fibers arranged in a bundle. Similarly, it is further possible to use optical waveguide(s), or photonic crystalline fiber (PCF)—also known as Holley fibers. These PCF or Holley fibers can be used since they can exhibit endlessly single mode properties over a wide wavelength ranges of light. Furthermore it is also possible to use double clad, triple clad, quadruple, or “many” clad fibers within the imaging probe 1 as well.

In one or more embodiments described herein, the motor 344, or component(s) of the motor 344 (such as a rotor), may be implemented inside the elongate member 300. Medical probes with internal rotor have been described in U.S. patent application Ser. Nos. 13/006,390 and 13/006,404, the disclosures of both of which are expressly incorporated by reference herein.

In the above embodiments, the probe 1 has been described as having a light source for imaging. In other embodiments, the probe 1 may have other components for providing other types of imaging. For example, in other embodiments, the probe 1 may include an ultrasound transducer for emitting acoustic signals. The ultrasound transducer may be coupled to a shaft located within the lumen 308 of the elongate member 300. The shaft may be coupled to the motor 344, which is configured to turn the shaft to thereby rotate the ultrasound transducer.

Although embodiments of the imaging probe 1 has been described as having an imaging function, in other embodiments, the imaging probe 1 may have treatment functionality. Thus, as used in this specification, the term “imaging probe” or similar terms, should not be limited to a device that can only performing imaging. For example, in other embodiments, the imaging probe 1 may be a laser surgical probe. In other embodiments, the probe 1 can transmit and receive optical radiation as previously described, but the probe 1 may also transmit optical energy having an energy that is enough to ablate tissue or cells within a narrow passageway such as an artery, vein, esophagus, colon, intestines, or other parts of the body. In any of the embodiments of the probe 1 described herein, the probe's detected optical radiation may be used by a processor as feedback to control the laser ablative source. The laser providing ablative power may be operated in constant wave (CW), pulsed, modelocked, or q-switched, or quasi-modelocked/q-switched.

Also, in further embodiments, the imaging probe 1 may be used outside the medical field. For example, in other embodiments, the imaging probe 1 may be an industrial inspection probe. In such cases, the probe 1 may be used to examine and ablate materials inside narrow passage ways, such as machine bores and holes, or to perform inspection of different objects.

Also, it should be noted that although embodiments of the probe 1 have been described as having imaging capability, in other embodiments, the probe 1 may be configured to perform treatment. For example, in other embodiments, the light beam provided by the probe 1 may have an energy level that is sufficient to treat tissue (e.g., for ablation). Also, in other embodiments, instead of coupling one or more optical components to the motor 344, the probe 1 may include an energy delivery device that is coupled to the motor 344, thereby allowing the energy delivery device to be rotated by the motor 344. By means of non-limiting examples, the energy delivery device may be an ultrasound transducer, a heat emitting device, etc.

Although particular embodiments have been shown and described, it will be understood that they are not intended to limit the present inventions, and it will be obvious to those skilled in the art that various changes and modifications may be made without departing from the spirit and scope of the present inventions. The specification and drawings are, accordingly, to be regarded in an illustrative rather than restrictive sense. The present inventions are intended to cover alternatives, modifications, and equivalents, which may be included within the spirit and scope of the present inventions as defined by the claims.

What is claimed:

1. A medical device, comprising:
an elongate member having a proximal end, a distal end, a body extending between the proximal end and the distal end, and a lumen located within the body;
a tube located inside the lumen, wherein the tube is rotatably supported inside the lumen;
an optical waveguide located inside the tube; and
a bearing element located in the lumen and disposed between the tube and the body of the elongate member;
wherein the elongate member further has a side wall and a region at the side wall for allowing an output light from the optical waveguide to exit therethrough.

2. The device of claim 1, wherein the bearing element comprises a first bearing and a second bearing that are disposed along a longitudinal length of the elongate member.

3. The device of claim 1, wherein the bearing element comprises a bearing that is fixedly secured to an outer surface of the tube.

4. The device of claim 1, wherein the bearing element comprises a bearing that is fixedly secured to an inner surface of the body of the elongate member.

5. The device of claim 4, wherein the bearing element comprises a bearing having a thickness that varies as a function of radial distance measured from the inner surface of the body.

6. The device of claim 1, wherein the bearing element comprises a first ring.

7. The device of claim 6, wherein the bearing element comprises a second ring, and wherein the first and second rings are at different locations along a longitudinal length of the elongate member.

8. The device of claim 6, wherein the first ring has a thickness that varies as a function of distance measured radially from the tube.

9. The device of claim 1, wherein the bearing element comprises a first group of supports that is disposed circumferentially around the tube.

10. The device of claim 9, wherein the bearing element comprises a second group of supports that is disposed circumferentially around the tube, and wherein the first group and the second group are at different locations along a longitudinal length of the elongate member.

11. The device of claim 9, wherein each of the supports has a thickness that varies as a function of distance measured radially from the tube.

12. The device of claim 1, wherein the tube has a cross sectional dimension that varies along a longitudinal length of the tube in a periodic manner.

13. The device of claim 1, wherein the bearing element comprises a plurality of spheres.

14. The device of claim 13, wherein the spheres are loosely packed inside the lumen between the body of the elongate member and the tube.

15. The device of claim 13, wherein the plurality of spheres comprises a first group of spheres and a second group of spheres, and wherein the device further comprises a partition located inside the lumen for separating the first and second groups of spheres.

16. The device of claim 13, further comprising a blocker located inside the lumen and proximal to the region for preventing the spheres from moving distally.

17. The device of claim 13, wherein the spheres has a fill percentage that is anywhere from 0.01% to 97%.

18. The device of claim 1, wherein the elongate member is flexible.

19. The device of claim 1, wherein the elongate member, the tube, the fiber, and the bearing element are parts of an OCT system.

20. The device of claim 1, further comprising an optical component aligned with the optical waveguide at the distal end of the elongate member for directing the output light from the optical waveguide to exit laterally through the region of the elongate member.

21. A medical device, comprising:
a flexible elongate member having a proximal end, a distal end, a body extending between the proximal end and the distal end, and a lumen located within the body;
a tube located inside the lumen, wherein the tube is rotatably supported inside the lumen;
an optical waveguide located inside the tube; and
a plurality of spheres located in the lumen and disposed between the tube and the body of the elongate member;
wherein the elongate member further has a side wall and a region at the side wall for allowing an output light from the optical waveguide to exit therethrough.

22. The device of claim 21, wherein the spheres are loosely packed inside the lumen between the body of the elongate member and the tube.

23. The device of claim 21, wherein the plurality of spheres comprises a first group of spheres and a second group of spheres, and wherein the device further comprises a partition located inside the lumen for separating the first and second groups of spheres.

24. The device of claim 21, further comprising a blocker located inside the lumen and proximal to the region for preventing the spheres from moving distally.

25. The device of claim 21, wherein the spheres has a fill percentage that is anywhere from 0.01% to 97%.

26. The device of claim 21, further comprising an optical component aligned with the optical waveguide at the distal end of the elongate member for directing the output light from the optical waveguide to exit laterally through the region of the elongate member.

27. A medical device, comprising:
an elongate member having a proximal end, a distal end, a body extending between the proximal end and the distal end, and a lumen located within the body;
a tube rotatably supported inside the lumen, wherein the tube has a cross sectional dimension that varies along a longitudinal length of the tube in a periodic manner; and
an optical waveguide located inside the tube;
wherein the elongate member further has a side wall and a region at the side wall for allowing an output light from the optical waveguide to exit therethrough.

28. The device of claim 27, further comprising a bearing element disposed between the body of the elongate member and the tube.

29. The device of claim 27, wherein the tube has a plurality of large cross sections that interface with an inner surface of the body of the elongate member, and a plurality of small cross sections that interface with the optical waveguide inside the tube.

30. The device of claim 27, further comprising an optical component aligned with the optical waveguide at the distal end of the elongate member for directing the output light from the optical waveguide to exit laterally through the region of the elongate member.
